SEQUENCE LISTING (1 sequence)

SEQ ID NO: 1 - DNA, 6703 bp, synthetic construct
  source 1..6703: mol_type = other DNA; note = anti-Vimentin IgG recombinant plasmid, nucleotide sequence
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcctgacacgaagcttgccgccaccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaagggggtggtggctctcaccaccaccaccaccactaaaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2026/0049995 A1
(43) Pub. Date: Feb. 19, 2026

(54) USE OF CEREBROSPINAL FLUID-DERIVED ANTI-VIMENTIN IGG AS MARKER IN DIAGNOSIS OF VIMENTIN ANTIBODY-ASSOCIATED ASTROCYTOPATHY

(71) Applicant: Dongshan WAN, Luoyang (CN)

(72) Inventor: Dongshan WAN, Luoyang (CN)

(21) Appl. No.: 18/805,271

(22) Filed: Aug. 14, 2024

(51) Int. Cl. G01N 33/68; G01N 33/58

(52) U.S. Cl. CPC G01N 33/686; G01N 33/582; G01N 33/6893; G01N 2333/46; G01N 2800/24

(57) ABSTRACT

Use of a cerebrospinal fluid (CSF)-derived anti-Vimentin IgG antibody as a marker in diagnosis of Vimentin antibody-associated Astrocytopathy (VIMA) is provided, belonging to the technical field of biomedicine. It is proposed for the first time that the CSF-derived anti-Vimentin IgG can be used as the marker for diagnosing the VIMA and for preparing a product for diagnosing the VIMA. Use of a reagent for detecting a CSF-derived anti-Vimentin IgG in preparation of a product for diagnosing VIMA is provided for the first time. The anti-Vimentin IgG as the marker of the VIMA has a clinical sensitivity of 100% and a clinical specificity of 99% for diagnosing the VIMA.

### REFERENCE TO SEQUENCE LISTING

A computer readable XML file entitled “GWP20240503818-SEQUENCE LISTING”, which was created on Aug. 2, 2024, with a file size of about 8,662 bytes, contains the sequence listing for this application, has been filed with this application, and is hereby incorporated by reference in its entirety.

### TECHNICAL FIELD

The present disclosure belongs to the technical field of biomedicine, and in particular directs to use of a cerebrospinal fluid (CSF)-derived anti-Vimentin IgG as a marker in diagnosis of Vimentin antibody-associated Astrocytopathy (VIMA).

### BACKGROUND

Specific autoantibodies are closely associated with autoimmune diseases of the central nervous system (CNS) and play an important role in the diagnosis of such diseases. The diagnosis of autoimmune astrocytopathy which has gradually been recognized in recent years mainly relies on the detection of autoantibodies specific to astrocytes. However, in clinical practice, the specific autoantibodies required for diagnosis in some cases showing positive fluorescence on the astrocytes are lacking.

Autoantibodies specific against different neural cell antigens may exist in CSF or serum, and are closely related to autoimmune diseases of the CNS. However, the etiology of many patients with encephalitis or myelitis is unclear or no specific biomarkers (including autoantibodies) for diagnosis can be found in clinical practice, which may delay the diagnosis and treatment of the diseases. Autoimmune glial fibrillary acidic protein (GFAP) astrocytopathy (GFAP-A) is a recently discovered autoimmune meningoencephalomyelitis, but there are no studies or reports on VIMA so far.

### SUMMARY

In view of this, an objective of the present disclosure is to provide use of a CSF-derived anti-Vimentin IgG as a marker in preparation of a product for diagnosing VIMA.

Another objective of the present disclosure is to provide use of a reagent for detecting a CSF-derived anti-Vimentin IgG in preparation of a product for diagnosing VIMA.

Another objective of the present disclosure is to provide a cell culture plate for immunoglobulin (Ig) G anti-Vimentin IgG detection and a preparation method thereof.

Another objective of the present disclosure is to provide a kit for detecting a CSF-derived anti-Vimentin IgG and a diagnostic kit for VIMA.

To achieve the above objectives, the present disclosure provides the following technical solutions:

The present disclosure provides use of a CSF-derived anti-Vimentin IgG as a marker in preparation of a product for diagnosing VIMA.

The present disclosure further provides use of a reagent for detecting a CSF-derived anti-Vimentin IgG in preparation of a product for diagnosing VIMA.

The present disclosure further provides a cell culture plate for anti-Vimentin IgG detection, where the cell culture plate is coated with cells transfected with an anti-Vimentin IgG recombinant plasmid, and the anti-Vimentin IgG recombinant plasmid has a nucleotide sequence set forth in SEQ ID NO: 1.

In some embodiments, the cell includes a eukaryotic adherent expression cell.

The present disclosure further provides a preparation method of the above cell culture plate, including the following steps: treating a surface of a cell-bearing substrate with a cell adhesion reagent, inoculating the cell to allow culture; when a cell density reaches 50% to 70%, transfecting the cell with the anti-Vimentin IgG recombinant plasmid and a transfection reagent; and fixating the transfected cell with a fixative, and washing to obtain the cell culture plate for anti-Vimentin IgG detection.

The present disclosure further provides a kit for detecting a CSF-derived anti-Vimentin IgG, including the above cell culture plate.

The present disclosure further provides a diagnostic kit for VIMA, including a fluorescently-labeled anti-human IgG secondary antibody and the above cell culture plate.

In some embodiments, if the anti-Vimentin IgG in a CSF of a subject to be detected is positive and has a titer of ≥1:10 (which means that it is still positive after being diluted for more than 10 times), the subject is considered to be suffered from an VIMA; if the anti-Vimentin IgG in the CSF of a subject to be detected is negative, the subject is considered to be not suffered from the VIMA; and if the anti-Vimentin IgG in the CSF of a subject to be detected is positive but has a titer of <1:10 (i.e., negative after being diluted more than 10 times), the subject is considered to be not suffered from the VIMA.

The present disclosure has the following beneficial effects:

In the present disclosure, a newly discovered autoimmune meningoencephalomyelitis, designated “Vimentin antibody-associated Astrocytopathy (VIMA)”, is defined and described, which expands the scope of autoimmune astrocytopathy and is helpful for the early diagnosis of autoimmune meningoencephalomyelitis in clinical practice.

It is proposed for the first time that the CSF-derived anti-Vimentin IgG can be used as a marker for diagnosing the VIMA and for preparing a product for diagnosing the VIMA. Use of a reagent for detecting a CSF-derived anti-Vimentin IgG in preparation of a product for diagnosing VIMA is also provided for the first time. Using the anti-Vimentin IgG as a marker of the VIMA for diagnosing the VIMA has a clinical sensitivity of 100% and a clinical specificity of 99%.

In the present disclosure, the cell culture plate for anti-Vimentin IgG detection may be used to accurately detect whether a CSF to be detected contains the anti-Vimentin IgG and determine a titer of the anti-Vimentin IgG against the Vimentin, and may further be used to accurately diagnose the VIMA, which provides a new perspective for the clinical diagnosis and treatment of autoimmune diseases of the CNS.

### BRIEF DESCRIPTION OF THE DRAWINGS

FIG. 1A-FIG. 1B show the image by magnetic resonance imaging (MRI) scans and immunostaining of 2 patients with unknown encephalomyelopathy, where FIG. 1A is an image showing the MRI scan results of symmetrical lesions in the bilateral corticospinal tracts, spinal cord, and cerebellum of the 2 patients, and the lesions (arrows) are shown by T2 or T2 fluid attenuated inversion recovery (FLAIR); and FIG. 1B is an image showing the immunostaining results of the CSF of the 2 patients on the cerebellum and spinal cord tissue of a rat, where Bar=50 μm; and

FIG. 2A-FIG. 2B show the results of cell response in Example 3, where FIG. 2A is a gradient dilution result of unknown encephalomyelopathy patient #1; and FIG. 2B is the result corresponding to a 1:3.2 dilution, where Bar=50 μm.

### DETAILED DESCRIPTION OF THE EMBODIMENTS

The present disclosure provides use of a CSF-derived anti-Vimentin IgG as a marker in preparation of a product for diagnosing VIMA.

In the present disclosure, the VIMA is a self-defined new disease, which is not rare among clinically undiagnosed autoimmune diseases of the CNS. Specifically, the VIMA accounts for 3.4% of clinically undiagnosed autoimmune diseases of the CNS. The clinical manifestations of VIMA are: subacute/progressive meningoencephalomyelitis, characterized by meningeal signs, tremor, cerebellar ataxia, brainstem manifestations, weakness, and corticospinal tract signs; the characteristic imaging manifestations of VIMA are: bilateral corticospinal tract abnormal signals, patchy abnormal signals in the white matter, cerebellum, and brainstem, and discontinuous and blurred lesions in long segments of the spinal cord. Most patients have elevated levels of protein and mononuclear cells in their CSFs. At the same time, increased intrathecal IgG synthesis rate or CSF-specific oligo clonal band (OCB) occurs. Patients may or may not be accompanied by peripheral nerve damage.

In the present disclosure, VIMA and GFAP-A have overlapping clinical symptoms, both show similar fluorescence patterns based on tissue substrates, but different clinical features. Patients with VIMA have a higher incidence of cerebellar ataxia, brainstem involvement, tremor, corticospinal tract lesions and the like, while patients with GFAP-A are more likely to have encephalopathy symptoms such as seizures, confusion, altered consciousness, perivascular linear radiating enhancement and the like. In addition, VIMA has a higher recurrence rate and MRI lesions are generally non-enhanced. The coexistence of anti-Vimentin IgG and anti-GFAPα IgG in CSF suggests a more extensive spectrum of autoimmune astrocytopathy. Treatment options for GFAP-A may also be applicable to the VIMA, although personalized adjustments in medication dosage, such as steroid dose and duration of maintenance therapy, may be required.

The present disclosure further provides use of a reagent for detecting a CSF-derived anti-Vimentin IgG in preparation of a product for diagnosing VIMA. In the present disclosure, the product type includes a kit.

The present disclosure further provides a cell culture plate for detecting anti-Vimentin IgG, where the cell culture plate is coated with cells transfected with an anti-Vimentin IgG recombinant plasmid, the anti-Vimentin IgG recombinant plasmid has a nucleotide sequence set forth in SEQ ID NO: 1, and the nucleotide sequence set forth in SEQ ID NO: 1 is a eukaryotic plasmid containing a coding gene sequence for the amino acid sequence of the protein.

In the present disclosure, the cell preferably includes a eukaryotic adherent expression cell, and the eukaryotic adherent expression cell preferably is a HeLa cell. The anti-Vimentin IgG recombinant plasmid is preferably obtained by inserting a human Vimentin cDNA into a pcDNA3.1 plasmid, and the human Vimentin cDNA is preferably obtained by amplifying from the HEK293T cell.

The present disclosure further provides a preparation method of the cell culture plate, including the following steps: treating a surface of a cell-bearing substrate with a cell adhesion reagent, inoculating the cell to allow culture; when a cell density reaches 50% to 70%, transfecting the cell with the anti-Vimentin IgG recombinant plasmid and a transfection reagent; and fixating the transfected cell with a fixative, and washing to obtain the cell culture plate for anti-Vimentin IgG detection. In the present disclosure, the fixative is 1% paraformaldehyde.

In the present disclosure, the cell-bearing substrate preferably includes a microwell plate or a glass slide; there is no special limitation on the specific source of the microwell plate or glass slide. The cell adhesion reagent preferably includes poly-D-lysine (PDL) or gelatin. When a surface of the cell-bearing substrate is treated with the PDL, the PDL preferably has a concentration of 50 μg/mL. The cell to be inoculated preferably includes a eukaryotic adherent expression cell, the eukaryotic adherent expression cell preferably includes a HeLa cell, and the cell is preferably inoculated at a density of 30% to 50%. The culture is preferably conducted at 37° C. and 5% CO₂, and the transfection reagent is preferably M5 Hiper Lipo2000 reagent. The anti-Vimentin IgG recombinant plasmid and the transfection reagent are used at a volume ratio of preferably 1:2.5.

The present disclosure further provides a kit for detecting a CSF-derived anti-Vimentin IgG, including the above cell culture plate.

The present disclosure further provides a diagnostic kit for VIMA, including a fluorescently-labeled anti-human IgG secondary antibody and the above cell culture plate. In the present disclosure, the fluorescent label is preferably Alexa Fluor 488. When VIMA diagnosis is conducted using the diagnostic kit for VIMA, if the anti-Vimentin IgG in a CSF of a subject to be detected is positive and has a titer of ≥1:10, the subject is considered to be suffered from VIMA; if the anti-Vimentin IgG in the CSF of a subject to be detected is negative, the subject is considered to be not suffered from the VIMA; and if the anti-Vimentin IgG in the CSF to be detected is positive but has a titer of <1:10, the subject is considered to be not suffered from VIMA. The titer determination method in the present disclosure includes: diluting a sample to be detected in PBS at ratios of 1:3.2, 1:10, 1:32, 1:100, 1:320, and 1:1000; and when a positive signal disappears after being diluted to a certain gradient, regarding a previous dilution ratio of the certain gradient as a sample titer.

The technical solutions provided by the present disclosure will be described in detail below with reference to the examples, which should not be construed as limiting the claimed scope of the present disclosure.

In the following examples, all methods are conventional methods, unless otherwise specified.

All materials and reagents used in the following examples are commercially available, unless otherwise specified.

### Example 1

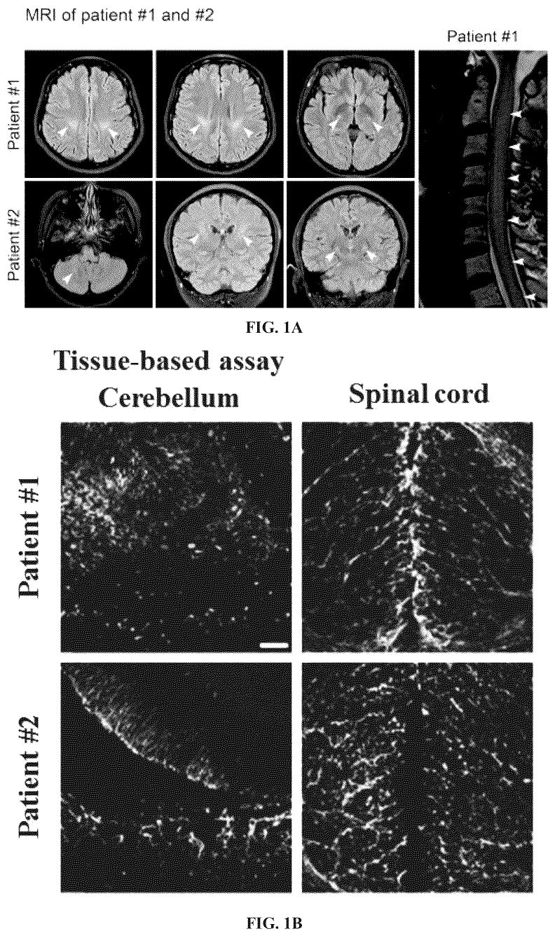

2 patients with unknown encephalomyelopathy (#1 and #2) (currently known to be negative for CNS autoantibodies detection) were recruited. Both patients showed clinical cerebellar ataxia or rhombencephalitis (or combined with myelitis). MRI showed bilateral corticospinal tract lesions (as shown in FIG. 1A), and astrocyte-like fluorescence positive of the CSF substrate, which was mainly distributed in the cerebellum, cerebellar Bergmann glial cells, ependymal cells around the lateral ventricles and fourth ventricle, and the central canal of the spinal cord and the subchondral area near the spinal cord pia mater (as shown in FIG. 1B). These observations suggested the possibility of a novel autoimmune astrocytopathy.

To elucidate the antibody origin of the astrocyte-like fluorescence observed by tissue substrate-based fluorescence experiments, 10×Genomics single-cell sequencing were conducted on the CSF samples of the 2 patients,

Plasmablast/plasma cell expressing clonotype or clonally expanded clonotype IgG (regardless of cell type) were further selected for subsequent analysis. A total of 33 clonotypes from 2 patients were expressed in vitro. The purified antibodies were then screened for expression in rat tissues to detect their autoreactivity. 13 monoclonal antibodies (mAbs) had fluorescence similar to those observed in patient tissue substrate-based fluorescence experiments.

The fluorescence images of the 13 mAbs were similar, mainly localizing to astrocytes in the molecular and granular layers of the cerebellum, ependyma and periependymal regions of the lateral ventricles, midbrain pia mater, basal ganglia, brainstem, and spinal cord gray and white matters. Astro #1_c5 (mAb number) had the most obvious fluorescence imaging, and some vascular structures were also found.

Target antigen identification was conducted by fluorescence assay based on a rat primary astrocyte substrate, where all 13 mAbs were reactive. Co-immunoprecipitation with Astro #1_c5 under non-reducing conditions pulled down two bands. The results of mass spectrometry analysis showed that band #1 matched with Vimentin with the highest score. Given the sequence similarity in the α-coiled-coil domain, the 13 mAbs were validated for their specificity against Vimentin and GFAPα proteins of a patient by cell-based immunofluorescence assay (CBA), and were found to be primarily reactive to Vimentin. The CSF of the 2 patients showed anti-Vimentin IgG positive based on cell-based immunofluorescence assay.

### Example 2

### Preparation of Cell Culture Plate for Anti-Vimentin IgG Detection:

1) Construction of anti-Vimentin IgG recombinant plasmid:

Human Vimentin cDNA was amplified from HeLa cells (ATCC) using a reverse transcription kit (Clontech Company) and inserted into a pcDNA3.1 plasmid using NEBuilder HiFi DNA Assembly Master Mix (NEB Company) to construct the anti-Vimentin IgG recombinant plasmid. The anti-Vimentin IgG recombinant plasmid had a sequence set forth in SEQ ID NO: 1.

2) HeLa cells were cultured in 10% FBS+DMEM high-glucose medium at 37° C. in a 5% CO₂ incubator; a surface of the cell culture plate was treated with 50 μg/mL PDL (Sigma Company). The HeLa cells (ATCC) were inoculated at a density of 30% in wells of the cell culture plate and cultured at 37° C. in a 5% CO₂ incubator.

3) When a cell density reached 50%, the cells were transfected with the anti-Vimentin IgG recombinant plasmid and M5 Hiper Lipo2000 reagent (Mei5 Biotech Company) at a ratio of 1:2.5.

4) Cell fixation: after 36 h, the cells were fixed with 1% PFA for 10 min and washed 3 times with 200 μL of PBS to obtain the cell culture plate for anti-Vimentin IgG detection.

### Example 3

The cell culture plate for anti-Vimentin IgG detection in Example 2 was used to detect whether the CSF samples of 2 index patients (#1 and #2) recruited in Example 1 and a patient who had been diagnosed with GFAP-A were anti-Vimentin IgG negative and the titer of the antibody, as follows:

The samples to be detected were diluted into 200 μL PBS at a ratio of 1:3.2, 1:10, 1:32, 1:100, 1:320, and 1:1000, respectively, which were added dropwise into the wells of the cell culture plate for anti-Vimentin IgG detection obtained in Example 2; the plate was incubated at 37° C. for 1 h in the dark; which was then washed 3 times with PBS; then an Alexa Fluor 488 Anti-human IgG secondary antibody (goat anti-human) diluted at 1:1000 in PBS was added into the wells and the plate was incubated for 1 h; which was then washed 3 times with PBS; a green fluorescence signal was observed under a 20× objective lens of a fluorescence microscope and the fluorescence images were captured. When the signal disappeared after dilution to a certain gradient, the dilution ratio of the upper level was regarded as a sample titer. At the same time, anti-GFAPα IgG from a GFAP-A patient was diluted at 1:3.2 as a control antibody to verify the specificity of the cell culture plate in Example 2.

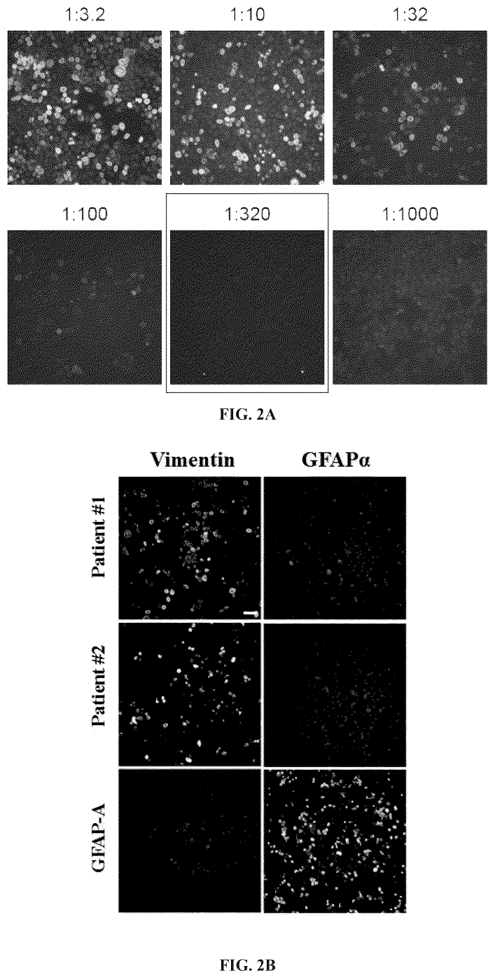

The results obtained by the above gradient dilution of unknown encephalomyelopathy patient #1 were shown in FIG. 2A, where the result corresponding to the dilution of 1:3.2 was shown in FIG. 2B. It was seen that unknown encephalomyelopathy patient #1 had a titer of ≥1:10; the CSF of the 2 unknown encephalomyelopathy patients (#1 and #2) recruited in Example 1 was anti-Vimentin IgG positive and anti-GFAPα IgG negative; while the CSF of the patient who had been diagnosed with GFAP-A was anti-Vimentin IgG negative and anti-GFAPα IgG positive. These results proved that the cell culture plate of the present disclosure had desirable specificity and high accuracy.

### Example 4

CSF samples were collected from a total of 610 patients admitted to the hospital for idiopathic inflammatory CNS demyelinating disease, cerebellar ataxia/encephalopathy/myelitis. In addition, CSF samples from 23 patients with autoimmune GFAP astrogliopathy (GFAP-A), 38 patients with NMDARE, 26 patients with NMOSD, 26 patients with MOGAD, 44 patients with MS, and 34 patients with cerebrovascular disease (CVD) were collected.

The cell culture plate for anti-Vimentin IgG detection in Example 2 was used to detect whether the CSF samples from the above patients were anti-Vimentin IgG positive and the titer of the anti-Vimentin IgG, as follows:

The samples to be detected were diluted into 200 μL PBS at a ratio of 1:3.2, 1:10, 1:32, 1:100, 1:320, and 1:1000, respectively, which were added dropwise into the wells of the cell culture plate obtained in Example 2 for anti-Vimentin IgG detection; the plate was incubated at 37° C. for 1 h in the dark; which was then washed 3 times with PBS; then an Alexa Fluor 488-Anti-human IgG secondary antibody (goat anti-human IgG) diluted at 1:1000 in PBS was added into the wells and the plate was incubated for 1 h; which was then washed 3 times with PBS; a green fluorescence signal was observed under a 20× objective lens of a fluorescence microscope and the fluorescence signal was captured. When the signal disappeared after dilution to a certain gradient, the dilution ratio of the upper level was regarded as a sample titer.

The results showed that 21 patients (3.4%) among the 610 patients (610 samples) were anti-Vimentin IgG positive. In which 2 patients (0.3%) also showed anti-Hu IgG positive and anti-Yo IgG positive in their serum, respectively. Of note, among the 23 patients with GFAP-A, 4 patients (17.4%) had definite anti-Vimentin IgG positivity in CSF. Among the 38 NMDARE patients, 2 patients (5.3%) showed definite anti-Vimentin IgG positivity (one of them had an anti-GFAPα IgG titer of 1:100). For 26 NMOSD patients, 26 MOGAD patients, 44 MS patients, and 34 CVD patients, their anti-Vimentin IgG was either negative or only low titer (<1:10) was observed.

### Example 5

CSF samples from 1,059 hospitalized patients with demyelinating diseases of the CNS, autoimmune encephalitis, and paraneoplastic neurological syndrome were collected during hospitalization. The cell culture plate for anti-Vimentin IgG detection in Example 2 was used to detect whether the CSF samples of the 1,059 patients was anti-Vimentin IgG positive and the titer of the anti-Vimentin IgG, the specific method was the same as that in Example 3. In addition, the CSF samples of the above patients were subjected to cell-based immunofluorescence experiments to detect autoantibodies. The currently known autoantibodies were screened including those associated with demyelination diseases (anti-Aquaporin-4 (AQP4) IgG, anti-myelin oligodendrocyte glycoprotein (MOG) IgG using CBA, autoimmune encephalitis (anti-N-methyl-D-aspartate receptor (NMDAR) IgG, anti-Leucine Rich Glioma Inactivated 1 (LGI1) IgG, anti-α-amino-3-hydroxy-5-methyl-4-isoxazolepropionic acid receptor (AMPAR) IgG, anti-γ-aminobutyric acid type B receptor (GABARB) IgG, anti-contactin-associated protein-2 (CASPR2) IgG, anti-immunoglobulin-like cell adhesion molecule 5 (IgLON5) IgG, anti-dipeptidyl-peptidase-like protein 6 (DPPX) IgG, anti-GFAPα IgG) using CBA, paraneoplastic syndrome (anti-Hu IgG, anti-Ri IgG, anti-Yo IgG, anti-CV2 IgG, anti-Ma2 IgG, anti-Amphiphysin IgG, anti-Recoverin IgG, anti-SRY-box transcription factor 1 (SOX1) IgG, anti-glutamic acid decarboxylase 65-kilodalton isoform (GAD65) IgG, anti-Delta/Notch-like epidermal growth factor-related receptor (DNER) IgG using immunoblot and gangliosides autoantibody (including GQ1b IgG/IgM)) using immunoblot.

The results showed that 29 samples (2.7%) were definitely positive for anti-Vimentin IgG. Among them, 11 patients (A total of 14 samples were collected from these patients, with 3 of them being sampled twice at different time) were exclusive positive for anti-Vimentin IgG, 6 patients (6 samples) were positive for anti-GFAPα IgG, 5 patients (5 samples) had infectious encephalitis (4/5 were detected with EBV), 1 tumor patient (1 sample) was positive for Yo autoantibody, and the other 3 patients (3 samples) were positive for anti-Vimentin IgG.

### Example 6

The titer of anti-Vimentin IgG antibody of 33 anti-Vimentin IgG positive patients from Examples 4 and 5 (21 anti-Vimentin IgG positive CSF samples out of 610 cases in Example 4+1 Yo antibody positive tumor patient in Example 4+11 patients (14 samples were collected from these patients, with 3 of them being sampled twice at different time) who only showed anti-Vimentin IgG positivity in Example 4) in Example 5 were detected, either alone (n=27) or in combination with low-titer anti-GFAPα IgG (n=2), and paraneoplastic antibodies (n=3). The results were shown in Table 1.

TABLE 1
Summary of clinical features and clinical
manifestations of 33 VIMA patients

| | Median [IQR] | |
|---|---|---|
| Items | or percent | |
| Clinical features | | |
| Age (years) | 41 | [27, 52] |

| | |
|---|---|
| Female | 61% |
| Relapse | 55% |
| First onset | |
| Subacute | 52% |
| Progressive | 48% |
| ICU admission | 0% |

Clinical manifestations

| Prodromal manifestations | 64% | |
|---|---|---|
| Meningeal irritation | 36% | |
| Consciousness decline | 13% | |
| Confusion | 4% | |
| Seizure | 0% | |
| Cognition decline | 25% | |
| Psychiatric symptom | 15% | |
| Tremor | 42% | |
| Cerebellar ataxia | 88% | |
| Nystagmus | 36% | |
| Vision loss | 27% | |
| Cranial nerve/Brainstem involvement | 85% | |
| Oculomotor dysfunction (III, IV, VI) | 42% | |
| Facial numbness (V) | 30% | |
| Hearing loss/Tinnitus (VIII) | 18% | |
| Facial/Tongue paralysis (VII, XII) | 18% | |
| Taste disturbances (VII, IX) | 3% | |
| Bulbar palsy (IX) † | 52% | |
| Weakness* | 70% | |
| Mild | 61% | |
| Moderate/Severe | 9% | |
| Abnormal superficial sensation | 64% | |
| Abnormal proprioception sensation | 27% | |
| Voiding dysfunction | 21% | |
| Pyramid signs | 88% | |
| PNS Involvement (by EMG/NCS) | 39% | (7/18) |
| Combined tumor | 3% | |
| Combined autoimmune diseases | 9% | |
| Radiological findings | | |
| Normal MRI | 12% | |
| Abnormal brain MRI | 70% | |
| Bilateral cerebral CST lesion | 36% | |
| Cerebral white matter lesion | 42% | |
| Infratentorial lesion | 27% | |
| Enhancement | 15% | |
| Abnormal spinal cord MRI | 42% | |
| 1-2 segments or punctate | 9% | |
| ≥3 segments | 33% | |
| Enhancement | 18% | |
| Combined brain & spinal cord MRI abnormality | 27% | |
| Meningeal enhancement | 6% | |
| Laboratory findings | | |
| Elevated CSF protein (>45 mg/dL) | 88% | |
| CSF protein (mg/dL) | 86.4 | [54.8, 112] |
| Elevated CSF WBC (>5 × 10\hat{ }6/L) | 85% | |
| CSF WBC (10\hat{ }6/L) | 36 | [9, 60] |
| Intrathecal synthesis | 94% | |
| Specific CSF OCB | 91% | |
| Elevated IgG SR (>9 mg/24 h) | 85% | |
| IgG SR (mg/24 h) | 46.61 | [14.67, 72.19] |
| CSF anti-VIM IgM titer ≥1:32 | 23% | (6/26) |
| Serum anti-VIM IgG titer ≥1:1,000 | 100% | (27/27) |
| Serum anti-VIM IgM titer ≥1:1,000 | 92% | (24/26) |
| Treatments and Follow-ups | | |
| Follow-up time (days) | 540 | [332, 760] |
| Maximum mRS | | |
| 5 | 3% | |
| 3-4 | 76% | |
| 0-2 | 21% | |
| Last follow-up mRS | | |
| 5 | 3% | |
| 3-4 | 52% | |
| 0-2 | 45% | |
| First-line therapy | | |
| Corticosteroid pulse | 82% | |
| IVIG | 30% | |
| Long-term therapy | | |
| Oral Corticosteroid | 82% | |
| MMF | 36% | |
| anti-CD20 | 9% | |
| Rapid improvement during first-line therapy‡ | 37% | (11/30) |
| Recovery in an episode# | 66% | (19/29) |

*MUSCLE STRENGTH AT 4/5 OR ABOVE IS CONSIDERED MILD.
† BULBAR PALSY INCLUDES ARTICULATION DISORDER AND DYSPHAGIA, DETERMINED BY WATER SWALLOW TEST, HYPOPHARYNGEAL REFLEX, TONGUE WEAKNESS
‡TREATMENT RESPONSE DEPENDS ON DOCUMENTED PHYSICAL EXAMINATION AND SELF-REPORT OF PATIENT.
#REDUCTION OF MRS IS MORE THAN 50%.
ABBREVIATIONS: VIM = VIMENTIN; PNS = PERIPHERAL NERVE SYMPTOMS; EMG = ELECTROMYOGRAPHY; CST = CORTICOSPINAL TRACT. NCS = NERVE CONDUCTION STUDY; WBC = WHITE BLOOD CELLS; SR = SYNTHESIS RATE; OCB = OLIGOCLONAL BANDS; IVIG = INTRAVENOUS IMMUNOGLOBULIN; MMF = MYCOPHENOLATE MOFETIL; MRS = MODIFIED RANKIN SCALE.

For a VIMA patient, anti-Vimentin IgG may also present in serum at a high level (i.e., titer≥1:320). However, serum anti-Vimentin IgG could also be detected in patients with other CNS autoimmune diseases, but its titer was low.

The median age of onset of the 33 anti-Vimentin IgG-positive patients was 41 years old, with no significant difference in gender. Among them, 48% of the patients showed progressive or step-by-step onset (from onset to peak for more than 2 months). Relapse occurred in 55% patients. Analysis of the clinical manifestations of these patients included: prodromal manifestations of infection or vaccination (64%), meningeal irritation signs (36%), low-frequency postural tremor of the head, upper limbs, and trunk (42%), cerebellar ataxia (88%), brainstem encephalitis (85%), movement disorders (70%), superficial sensory disorders (64%), corticospinal tract signs (88%), and peripheral nerve damage (39%). One of the patients had combined rectal cancer.

In terms of radiological findings, 64% of the patients that were anti-Vimentin IgG positive and had a titer of ≥1:10 showed abnormal brain MRI. Among them, 30% patients with abnormal brain MRI had characteristic manifestations of bilateral corticospinal tract lesions; 42% of the patients had brain white matter lesions; 27% of the patients had brainstem and cerebellar lesions; and 15% of the patients showed lesion enhancement. MRI showed spinal cord involvement in 42% of patients, mainly manifested as longitudinal discontinuous and blurred lesions (accounting for 78% of spinal cord lesions).

It could be seen that the patients that were anti-Vimentin IgG positive and had a titer of ≥1:10 showed similar clinical features and manifestations, radiological findings, and laboratory findings. Therefore, in the present disclosure, the patients that were detected to be anti-Vimentin IgG positive in CSF with a titer of ≥1:10 and had the above clinical and radiological characteristics were defined as “VIMA” patients.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for treating a Vimentin antibody-associated Astrocytopathy (VIMA) in a subject, comprising
detecting whether cerebrospinal fluid (CSF) to be detected in the subject contains an anti-Vimentin IgG using a reagent for detecting the anti-Vimentin IgG,
determining a titer of the anti-Vimentin IgG against the Vimentin,
diagnosing whether the subject is suffered from the VIMA according to the titer of the anti-Vimentin IgG against the Vimentin, and
treating the subject in need thereof according to a diagnosis result.

2. A cell culture plate for detecting an anti-Vimentin IgG, wherein the cell culture plate is coated with a cell transfected with an anti-Vimentin IgG recombinant plasmid, and the anti-Vimentin IgG recombinant plasmid has a nucleotide sequence set forth in SEQ ID NO: 1.

3. The cell culture plate according to claim 2, wherein the cell comprises a eukaryotic adherent expression cell.

4. A method for preparing the cell culture plate according to claim 2, comprising the following steps:
treating a surface of a cell-bearing substrate with a cell adhesion reagent;
inoculating a cell to allow culture;
transfecting the cell with an anti-Vimentin IgG recombinant plasmid and a transfection reagent when a cell density reaches 50% to 70%;
fixating the transfected cell with a fixative; and
washing to obtain the cell culture plate for anti-Vimentin IgG detection.

5. The method according to claim 4, wherein the cell comprises a eukaryotic adherent expression cell.

6. A kit for detecting a CSF-derived anti-Vimentin IgG, comprising the cell culture plate according to claim 3.

7. The kit according to claim 6, wherein the cell comprises a eukaryotic adherent expression cell.

8. A kit for diagnosing a VIMA, comprising the cell culture plate according to claim 3 and a fluorescently-labeled anti-human IgG secondary antibody.

9. The kit according to claim 8, wherein
if an anti-Vimentin IgG in a CSF of a subject to be detected is positive and has a titer of ≥1:10, the subject is determined to be suffered from the VIMA;
if the anti-Vimentin IgG in the CSF of a subject to be detected is negative, the subject is determined to be not suffered from the VIMA; and
if the anti-Vimentin IgG in the CSF of a subject to be detected is positive but has a titer of <1:10, the subject is determined to be not suffered from the VIMA.
